(19) United States
(12) Patent Application Publication
(10) Pub. No.: US 2010/0316690 A1
(43) Pub. Date: Dec. 16, 2010

(54) Modular assembly of tissue engineered constructs

(76) Inventors: Michael V. Sefton, Toronto (CA); Alison McGuigan, Toronto (CA)

Correspondence Address: RICHES, MCKENZIE & HERBERT, LLP, SUITE 1800, 2 BLOOR STREET EAST, TORONTO, ON, M4W 3J5, CA

(21) Appl. No.: 12/457,507

(22) Filed: Jun. 12, 2009

(51) Int. Cl. C12N 5/07; A61F 2/02

(52) U.S. Cl. 424/424; 435/395; 435/397; 435/366

(57) ABSTRACT

Scaleable, vascularised tissue constructs that are composed of a multiplicity of cell containing, discrete and separable modules, methods of fabricating same and uses thereof. The tissue construct is a tissue substitute used in tissue transplantation or substitution or for the purpose of in vitro mimic of normal tissue.

### FIELD OF THE INVENTION

This invention relates to scalable, vascularised tissue constructs that are comprised of a multiplicity of cell containing, discrete and separable modules, methods of fabricating the same, and uses thereof.

### BACKGROUND OF THE INVENTION

It is desirable to create an unlimited supply of vital organs, such as hearts, livers and kidneys, for example, for transplantation through tissue engineering. In the past, there have been many suggested approaches to tissue engineering. One fundamental difficulty in creating large three-dimensional organs is the creation of a vascularised support structure in the engineered tissue or tissue construct. A tissue construct is a tissue substitute for the purpose of tissue transplantation or substitution or for the purpose of in vitro mimic of normal tissue.

The prior art suggests that mammalian cells may be grown in culture and seeded into a porous scaffold^{1,2} embedded within a gel like collagen³ or fibrin, or encapsulated within a semi-permeable membrane⁴. All of these can lead to tissue constructs with different characteristic features, but in all cases there are constraints on nutrient, waste and oxygen diffusion that restrict construct size to that for which the viability and function of the cellular components can be supported by the limited rate of diffusion. For cell densities typical of tissues (10⁸ to 10⁹ cells/mL) this may be as low as 100 μm. To circumvent this issue it is necessary to vascularize the construct and, if the construct is to be implanted, to enable the internal vasculature to connect with that of the host so that blood containing nutrients and oxygen can perfuse through the entire construct and supply cells with these nutrients (and remove metabolic wastes), even those cells far away from the surface of the construct. Since blood needs to perfuse through the internal vasculature, it needs to consist of a material or structure that is demonstrably non-thrombogenic or the vasculature must be lined with endothelial cells which are exhibiting a non-activated nonthrombogenic phenotype. ¹Vacanti J P, Mor se M A, Saltzman W M, Domb A J, Perez-Atayde A, Langer R. Selective cell transplantation using bioabsorbable artificial polymers as matrices. J Pediatr Surg. 1988 January; 23(1 Pt 2):3-9²G. K. Naughton, B. A. Naughton, Three-dimensional cell and tissue culture system, U.S. Pat. No. Pat. 5,443,950, Aug. 22, 1995.³Bell E, Ivarsson B, Merrill C. Production of a tissue-like structure by contraction of collagen lattices by human fibroblasts of different proliferative potential in vitro. Proc Natl Acad Sci USA. 1979 March; 76(3):1274-8.⁴Uludag H, Sefton M V. Microencapsulated human hepatoma (HepG2) cells: in vitro growth and protein release J Biomed Mater Res. 1993 October; 27(10):1213-24.

It has been proposed by Mooney et al. (J. Control Rel., 64:91-102, 2000) to incorporate an endothelial cell mitogen (in this case, vascular endothelial growth factor, or VEGF) into three-dimensional porous poly(lactide-co-glycolide) (PLG) scaffolds during fabrication to promote scaffold vascularisation. Sustained delivery of bioactive VEGF translated into a significant increase in blood vessel ingrowth in mice and the vessels appeared to integrate with the host vasculature, as disclosed by Nor et al⁵. However, as described by Ahrendt et al. (Tissue Engineering, 4(2):117-130, 1998), VEGF is but one angiogenic factor and issues associated with the functional maturity of the vessels and the need for multiple factors may limit this strategy. ⁵Nor J E, Peters M C, Christensen J B, Sutorik M M, Linn S, Khan M K, Addison C L, Mooney D J, Polverini P J. Engineering and characterization of functional human microvessels in immunodeficient mice. Lab Invest; 81(4):453-63 (2001)

Because endothelial cells are the key element of physiological vasculatures, it is obvious to consider their use in preparing tissue constructs. Vacanti et al. (Tissue Engineering, 6:105-117, 2000; also Vacanti, U.S. Pat. No. 6,455,311, Sep. 24, 2002) proposed a hierarchical branched network mimicking the vascular system in two dimensions. Vacanti et al. etched silicon and Pyrex* surfaces with branching channels ranging from 10 μm to 500 μm in diameter, which were then seeded with rat hepatocytes and microvascular endothelial cells. This technique has been extended to a degradable polymer and flow through the channels was demonstrated using fluorescent microbeads, as disclosed by Terai et al. (Abstracts of the Third Biennial Meeting of the Tissue Engineering Society, Nov. 30-Dec. 3, 2000). The approach of Vacanti et al (Tissue Engineering, 6:105-117, 2000; also Vacanti, U.S. Pat. No. 6,455,311, Sep. 24, 2002) teaches the use of 2-D structures, but does not anticipate the creation of 3-D structures by assembly of endothelial covered modules.

Endothelial cells have been transplanted as a means of therapeutic angiogenesis. HUVEC, transfected with Bcl-2 (to inhibit apoptosis), suspended in a collagen-fibronectin gel and transplanted into the abdominal wall of immune compromised (SCID) mice developed into a complete microvascular bed with the HUVEC assuming the phenotype of arterial, venous and capillary EC⁶. Furthermore there was recruitment of mouse smooth muscle cells, so that chimeric vessels were created that augmented perfusion in a moderate hindlimb ischemia model. The mechanism of these effects is unclear but this study highlights the plasticity of EC and the remodeling that can occur upon transplantation. Koike et al⁷ have implanted similar gels containing HUVEC and mesenchymal precursor cells and the vascular network created by the transplanted cells integrated with the host vasculature and remained stable and functional for 1 year in vivo. Prevascularized skeletal muscle was also created⁸ in a PLGA scaffold by co-culturing skeletal muscle cells with HUVEC or human embryonic stem, cell derived endothelial cells and fibroblasts. It appeared that up to 40% of the human EC containing blood vessels “connected” to the host vasculature upon implantation and supported the viability of the engineered muscle, at least for 2 weeks. Taken together these studies indicate that transplanted (large vessel) EC can produce a functioning vasculature, connecting to that of the host. Rather than transplanting EC inside a collagen based gel or a conventional scaffold, we transplant EC on the surface of collagen (or synthetic polymer) gel as modules that create a vascularized tissue construct. ⁶Enis D. R., Shepherd B. R., Wang Y, Qasim A, Shanahan C. M., Weissberg P. L., Kashgarian M, Pober J S, Schechner J S, Induction, differentiation and remodeling of blood vessels after transplantation of Bcl-2 transduced endothelial cells, PNAS 102 (2): 425-430, 2005⁷N. Koike, D. Fukumura, O. Gralla, P. Au, J. S. Schechner, R. K. Jain, Tissue engineering: creation of long-lasting blood vessels, Nature 428, 138 (2004).⁸Levenberg, S., Rouwkema, J., Macdonald, M., Garfein, E. S., Kohane, D. S., Darland, D. C., Marini, R., van Blitterswijk, C. A., Mulligan, R. C., D'Amore, P. A., Langer, R. Engineering vascularized skeletal muscle tissue. Nat Biotechnol 23: 879-84(2005).

The endothelium comprises heterogeneous, metabolically active cells. There are considerable phenotypic differences between large and small vessel endothelial cells (EC), among different organs and even within the same organ, as disclosed by Hewett et al. (In Vitro Cell Dev Biol Anim, November; 29A(11):823-30, 1993). For example, growth characteristics of large and small vessel EC of the same organ vary on gelatin, as shown by Beekhuizen et al. (J Vasc Res, July-August; 31(4):230-9, 1994). These differences reflect environment differences: the extracellular matrix, paracrine/autocrine factors, cell-cell contact and biomechanical factors act as cues. Several counteracting mechanisms and factors (for instance, endothelin/NO, pericytes/TGFβ) likely cooperate to further regulate the maintenance of a quiescent phenotype under non-pathological conditions.

In normal tissue, the EC that line blood vessels and capillaries have a variety of roles in controlling vascular function. Secretion and surface expression of molecules such as nitric oxide (Palmer et al., Nature, 333:664-666, 1988), prostacyclin (Moncada, British J Pharm, 76:3-31, 1982) and endothelin, that act on smooth muscle cells, regulate vessel tone while those acting on leukocytes such as platelet-activating factor (McIntyre et al., J Clin Invest, July; 76(1):271-80, 1985), direct both initiation and progression of inflammation. Endothelial cells provide a haemocompatible surface by production of molecules that modulate platelet aggregation (such as prostacyclin, ADPase, von Willebrand Factor, vWF), coagulation (such as thrombomodulin, which regulates protein C, and tissue factor) and fibrinolysis (such as tissue plasminogen activator, tPA and, plasminogen activator inhibitor, PAI-1). Under normal physiological conditions, the endothelium has a non-thrombogenic phenotype but, depending on the local environment, the cell can be transformed into a pro-thrombotic surface, for example by the action of thrombin. A non-thrombogenic phenotype is characterized by prolonged whole blood clotting times, minimal platelet activation and platelet loss upon perfusion and the contribution to patent flow channels when exposed to whole blood.

Blood compatibility has been a consideration in the development of vascular grafts. Endothelial cells have been seeded on a variety of biologically compatible materials, with or without protein pre-coating with fibronectin, collagen and other proteins, as shown by Meinhart et al. (Ann Thorac Surg, 71:S327-31, 2001). Factors influential in the EC seeding technique have been identified as including cell source and isolation technique, method of cell deposition, EC adhesion to the graft under flow conditions, and the thrombogenicity of the EC, as described by Hedeman Joosten et al. (J Vasc Surg, 28:1094-1103, 1998). Pre-seeded cells may be lost on implantation due to insufficient adhesion, as shown by Williams (Cell Trans, 4(4):401-410, 1995), and thus the protection from thrombosis provided by the cells may be limited due to the incomplete cell coverage of the support structure. Various strategies have been explored to improve cell adhesion, as disclosed by Lin et al. (Biomaterials, 13(13):905-14, 1992); for example, precoating with adhesive protein as described by Vohra et al. (Br J Surg, April; 78(4):417-20, 1991), Schneider et al. (Clin Mater, 13(1-4):51-5, 1993) and Jarrell et al. (J Biomech Eng, May; 113(2):120-2, 1991). It is desirable to ensure that the EC remain adherent to the support structure, such that there are no bare spots, and that the EC maintain their antithrombogenic phenotype for proper vascularisation. The requirement for EC attachment means that the materials used for cell encapsulation (e.g., alginate⁹ or HEMA-MMA⁴) are not suitable for preparing vascularized constructs, since microcapsules are typically designed to have a surface that prevents cell attachment so as to minimize the fibrotic response on encapsulation. ⁹Lim F, Sun A M, Microencapsulated islets as bioartificial endocrine pancreas. Science. 1980 Nov. 21; 210(4472):908-10.

In the preparation of tissue constructs by seeding cells in a scaffold, it is often difficult to get cells deposited on the outside of a scaffold to migrate to the interior; typically they populate just the periphery of the scaffold, at best an outer millimetre or so. The initial cell distribution is not uniform and prior seeding approaches work best for small, two-dimensional constructs, such as for use in small animals, as disclosed by Burg et al. (J Biomed Mater Res, September 15; 51(4):642-9, 2000). However, this method does not scale well for larger constructs or larger animals. Some effort has been directed towards various dynamic seeding techniques, as discussed, for example, by Kim et al. (Bioeng, January 5; 57(1):46-54, 1998) and Vunjak-Novakovic et al. (Biotechnol Prog, March-April; 14(2):193-202, 1998). However the scalability of dynamic seeding techniques remains questionable. Hence it is desirable to create a means of creating tissue constructs which results in a uniform cell distribution and which is adequate to preparing both small and large constructs; i.e., a scalable process is desired. By scalable, we refer to the notion that the functional and morphological properties of a large construct can be inferred from the properties of a small construct. This implies that the underlying characteristics of both small and large constructs derive from the same physical and biological principles. Constructs prepared by methods that precede this invention are intrinsically different depending on whether a small or large construct has been prepared.

Embedding cells in a gel generates a uniform cell distribution. However, it does not address another limitation of current tissue constructs: the difficulty of mixing two or more different cells types together without the concern that the faster growing cell type will overtake the slower one. Layering one cell type over a different cell type, as in collagen gel vascular grafts as reported by Weinberg et al. (Science, January 24; 231(4736):397-400, 1986), circumvents this problem, but this method is not universally applicable. Accordingly, it is desirable to provide a tissue construct that allows for incorporation of two or more cell types that have different growth rates, and a means of fabricating the same.

### SUMMARY OF THE INVENTION

It is an object of this invention to overcome the disadvantages of the prior art. Also, it is an object of this invention to provide a new modular approach to the fabrication of tissue constructs that is scaleable, with a largely uniform cell distribution, and can accommodate multiple cell types and in which the porosity is created after cell incorporation or embedding. A further object of the invention is to create a vascularised tissue construct by seeding a construct containing tissue specific cells (such as liver cells, islets of Langerhans, cardiac muscle cells or fat cells) with endothelial cells.

The present invention resides in the porous structure that is created when an enclosure, that is for example, a column or tube or a tissue space, is filled with discrete, separable components, hereinafter referred to as modules and defined hereinafter. A random arrangement of said modules results in channels created from the interstitial spaces or voids among the modules which because of geometric constraints do not pack the entire enclosure or tissue space. The resulting plurality of interstitial spaces are interconnected such that there are interconnected channels throughout the assemblage of modules (the construct), which results in the construct being porous and perfuseable with fluid.

The packing may be arranged in the same manner as random packed columns found in chemical engineering process equipment or in chromatography/gel filtration columns. Preferably, the packing is chosen so that the channels in such columns are narrow, resulting in relatively high surface area and high mass transfer coefficients for a fluid that may pass through the porous packing (i.e., across the enclosure). Accordingly, such columns are efficient separating devices. This arrangement is advantageously adapted in the present invention for tissue engineering. Such assemblages of cell containing modules have been used as bioreactors for the production of molecules of biotechnological interest¹⁰ but they have not hitherto been considered as tissue constructs. ¹⁰J. Emami, N. Kondo, T. Takano, K. Suzuki, Methgods for large-scale cultivation of animal cells and for making supporting substrata for the cultivatio, U.S. Pat. No. 5,264,359 Nov. 23, 1993.

In this sense, a tissue construct is a new tissue substitute that is assembled from a multiplicity of discrete and separable modules. The resulting construct has adequate porosity to enable perfusion with a fluid. Preferably, the construct will have a porosity of 0.3 to 0.99, where the porosity is defined as the ratio of the volume of interstitial space to the volume of the enclosure or tissue space. The construct will have dimensions ranging from a mm to several cm and has the tissue specific function(s) of the cells embedded within the discrete modules (the “discrete” phase). The construct has two phases, a discrete phase representing the modules and a continuous phase representing the voids, interstitial spaces and interconnected channels. Tissue specific cells may also be included in the pores as in conventional scaffolds (e.g., Naughton and Naughton²) but this is an additional embodiment. Similarly filling the channels of the construct (the “continuous” phase) with a material that contains cells (as per Zdrahala and Zdrahala¹¹)is an additional embodiment. ¹¹R. J. Zdrahala and I. J. Zdrahala, in vivo tissue engineering with biodegradable polymers, U.S. Pat. No. 6,376,742, Apr. 23, 2002

Modules are the discrete and separable units that are assembled to form the tissue construct. They contain tissue-specific cells embedded within a material that provides the three dimensional discrete and separable form to the modules. A preferred shape is a cylinder, but spheres and yet more complex shapes are possible. These modules are less than a mm, and preferably less than 500 μm and even more preferred less than 250 um in critical dimension. The critical dimension is the diameter of cylindrical or spherical modules or the thickness of planar modules. More generally the critical dimension is the diffusion distance normal to the axis of the channel, when the modules are assembled to form a construct. The critical dimension is obvious to someone skilled in the art. Even in the critical dimension, the modules are not a monolayer of cells, in distinction to what is taught in Vacanti et al (Tissue Engineering, 6:105-117, 2000; also Vacanti, U.S. Pat. No. 6,455,311, Sep. 24, 2002).

Modules represent an intermediate level of structure between that of a cell or cell aggregate and that of a tissue construct. Filling an enclosure with cells or cell aggregates produces a large aggregate of agglomerated cells, of the dimensions of the enclosure. This does not yield the porous, perfuseable structure that is a tissue construct. Thus a key aspect of the module is the presence of the cell compatible material that provides adequate dimensional stability to the module so that upon filling the enclosure, the interconnected channels are preserved. While each module can be considered a functional tissue unit on its own, the intent is to create a larger scale functional structure (the construct) upon assembly of a multiplicity of modules, in an enclosure or a tissue space. The construct then displays a set of functional characteristics that combine, build upon and extend the functional characteristics of the discrete modules.

In an alternative embodiment discrete and separable modules are prepared as non-agglomerating cell aggregates that are assembled to form the tissue construct. Here aggregates are produced without an embedding material but in such a way that each aggregate repels the others and prevents their agglomeration into a large, non perfuseable construct.

Modules can consist of any tissue-specific cell or tissue-specific cell aggregate (e.g., spheroids) or tissue fragment (e.g., Islets of Langerhans) embedded within a homogeneous gelatinous material such as collagen or gelatin. In an alternative embodiment, the module can have its own internal porosity, at a smaller scale than the porosity associated with the assembly of a plurality of modules into a construct. Thus modules can be formed by suspending cells into a matrix material as a liquid which is subsequently solidified or the module can be preformed as a single porous entity and then filled with a multiplicity of cells as is done using conventional scaffolds for tissue engineering. In yet another embodiment, modules can also be formed by encapsulating cells in an appropriate material, so that cells are suspended in an aqueous phase in the core of the capsule and the material is used to form the semi-permeable shell. While there are many methods to produce microcapsules, the prior art teaches that these microcapsules are intended to be used only as separable units; the prior art does not intend that they be used after assembly as a tissue construct.

The shape of the module determines the porosity of the construct. For example, cylindrical modules are a preferred embodiment, instead of simpler, spherical ones, because of the greater porosity of randomly packed rods instead of spheres when placed inside an enclosure. The effect of aspect ratio on packing density is well known in the fiber composite materials and chemical engineering literatures. At an aspect ratio, the (L/D) of 5 the porosity of randomly packed cylinders is ˜0.7, instead of the ˜0.5 that is obtained with spheres. This porosity is necessary to provide space for subsequent cell seeding (see below) and to lower the pressure drop through the module filled construct. Preferably, the modules are a geometric shape selected to enable a packing of a predetermined porosity. While the characteristic dimension is chosen on the basis of diffusion distance, as described above, a module may measure from 10 μm to 20 m, along the longest axis of the module with the preferred lengths being 100 μm to 1 cm. The aspect ratio (length to lateral dimension) may vary from 1 to 1000 or even greater. Shapes more complex than a filled cylinder may generate yet higher porosities, as is well known in the chemical engineering literature. Hollow cylinders (cf, Raschig rings) or saddle-shapes (cf., Berl or Intalox™ saddles) are examples of more complex shapes that could be used to great benefit here. The technology for making complex shapes using microlithography methods is emerging¹². ¹²D. Dendukari, D. C. Pregibon, J. Collins, T. A. hatton, P. S. Doyle, Continuous flow lithography for high throughput microparticle synthesis. Nature Materials, 5, 365-369 (2006).

It will be understood to a person skilled in the art that the choice of geometric shape or size for the module will affect the fluid flow regime throughout the enclosure. For instance, pressure drop across the enclosure and shear forces to which the cells coating the modules are exposed will be influenced by the choice of geometric shape or size. For example, constructs consisting of randomly packed cylindrical rods result in greater porosity (and hence, lower pressure drop/shear forces) than would be obtained with spheres. Similarly, larger cylindrical rods will result in greater pore sizes than smaller cylindrical rods. Accordingly, the present invention includes the use of modules of any geometric shape or size to achieve the desired fluid flow characteristics throughout the enclosure.

Further, it may be desirable to use two or more different irregular and/or geometric shapes or sizes in combination, randomly distributed within in the enclosure, to achieve different porosities and flow regimes throughout the enclosure. Examples of geometric shapes may include, but are not limited to, cylinders, rods of hexagonal cross-section, rods of maltese cross-section, spheres, spheroids, ellipsoids, cones, conoids, tetrahedrons, cuboids, prisms, pyramids, frustums, wedges, toruses, toroids, hexahedrons, octahedrons, dodecahedrons, rhombohedrons and trapezohedrons.

In a preferred embodiment the modules are additionally covered with endothelial cells such that interstitial spaces remain once the modules fill an enclosure, so that the resulting interconnected channels are lined by the endothelial cells. Preferably, the endothelial cells do not completely fill the interstitial spaces between the modules, and the resulting interconnected channels remain large enough to allow fluid flow through the channels. Accordingly, fluid perfuses around the modules thereby allowing mass transfer between the fluid and the endothelial cells and ultimately between the fluid and the cells within the modules. Although the modules fill in an enclosure such that a porous structure results, the porosity by itself will not preserve cell viability if the tissue construct is beyond a size where diffusion distances become too large. Larger tissue constructs require an internal vascularised structure that preferably has nonthrombogenic surfaces. Accordingly, a preferred embodiment of the tissue construct of the present invention includes endothelial cells covering the modules to create a “pseudo-capillary” network (i.e. the interconnected channels) capable of supporting blood perfusion through the channels of the tissue construct.

The module material consists of a cell compatible material that provides dimensional stability to the module and prevents agglomeration of the tissue-specific cells into a single cellular mass without interconnected, perfuseable channels. It serves to keep the modules discrete and separable. It also makes the module more rigid and easier to handle, preferably without compromising cell viability. The module material must be permeable to nutrients, oxygen and waste products so that cells deep inside the modules are able to survive. It is preferable, however that the tissue-specific cells are not able to escape from the interior of the module. Examples of cell compatible material may include, but are not limited to, agarose, alginate, collagen, polyacrylates, synthetic polymers that are substantially stable and known to be biocompatible in vivo.

Semi-synthetic materials such as collagen-poloxamine¹³ and related materials¹⁴ or other polyethylene glycol based materials that can be photo-crosslinked in place are also useful as modular materials. Alternatively biodegradable materials such, as gelatin can be used. If endothelial cells are to cover the modules, as in the preferred embodiment, then the module material must enable the adherence of endothelial cells to its surface. Cell containing modules made from one material may be coated with a second material (e.g., collagen) or a protein (e.g., fibronectin) to enable the attachment of the covering endothelial cells. Since the covering cells must express a nonthrombogenic phenotype, preferred coating or module materials are those such as collagen that can enable this phenotype. Cross-linking agents can be used to produce stiffer, easier to handle modules, without compromising cell viability. Examples of cross-linking agents may include, but are not limited to, polyepoxide, carbodiimide, genipin or glutaraldehyde. ¹³A. Sosnik A. and M. V. Sefton, Semi-synthetic collagen/poloxamine matrices for Tissue Engineering, Biomaterials, 26, 7425-7435 (2005)¹⁴Sosnik A. and Sefton M. V., Methylation of poloxamine for enhanced cell adhesion, Biomacromolecules, 7, 331-338 (2006)

The enclosure which forms the construct and contains the plurality of modules may be the walls of a tissue cavity (e.g., an omental pouch or a subcutaneous pocket) in which the modules are implanted directly. Alternatively the enclosure is a separate tube or box or any other suitable shape to which modules can be added The dimensions of the enclosure define the size of the tissue construct and may measure from 0.1 mm to 1000 mm, as measured along the longest axis of the enclosure, and the preferred dimensions are 0.5 mm to 10 cm. Preferably, the modules include sufficient structural rigidity and strength to allow their packing in the enclosure without deformation and compaction.

In a preferred embodiment of the invention, tissue-specific cells (such as liver cells, islets of Langerhans, cardiac muscle cells or fat cells) are embedded in short collagen gel cylinders rods or spheres, preferably cylinders of 50 to 500 μm diameter and a length of 250 μm to 2 mm (aspect ratios of 1 to 1 (length to diameter) to 5 to 1), onto which endothelial cells, for example, human umbilical vein endothelial cells (HUVEC), can adhere. These collagen cylinders are preferably randomly packed into an enclosure such as a tube that may measure 1 mm to 100 cm, as measured along the longest axis of the tube, to form a tissue construct. The construct further includes interstitial spaces that are interconnected to form channels such that the construct is porous and perfuseable with fluid. Preferably, the porosity and channel size are sufficiently large and the endothelial cells adequately nonthrombogenic to allow whole blood to percolate around the modules and through the channels. Preferably, the collagen modules include sufficient structural rigidity and strength to allow their packing in the enclosure without deformation of the modules and without allowing the encapsulated tissue specific cells to grow beyond the boundaries of the modules.

In another embodiment, the invention includes randomly packed larger diameter modules proximal and distal to the “pseudo-capillary” bed in the enclosure. Accordingly, the invention provides for means to produce a more physiological branching hierarchy. In a further embodiment, the invention resides in creating constructs from mixtures of modules with embedded cells that consist of different cell types. Advantageously, because the modules prevent cell growth beyond their confines, incorporation of multiple cell types within a single construct is possible without compromising the viability of the embedded cells. Accordingly, the invention provides for multiple modules containing different cells to form a mixed cell tissue construct. In another embodiment, the embedded cells are genetically manipulated to enhance cell survival or function. Further, the cells may have specialised cell functions for example, HepG2 spheroids for ‘liver-like’ function.

In yet another aspect, the present invention provides for a method of manufacturing the tissue construct wherein the endothelial cells that cover the modules may be introduced to incomplete modules prior to their insertion into the enclosure. In an alternative embodiment, endothelial cells may be introduced into the enclosure to coat the modules after introduction of the incomplete modules into the enclosure. In either embodiment, the module is “complete” as having a first cell type enclosed in a suitable material of a suitable geometric shape, onto which a second cell type adheres to cover the geometric shape. However, an incomplete module may still be useful. The interstitial gaps between the module form interconnected channels that are lined by cells, regardless of the method of cell introduction. The resulting cell lining preferably enables fluid flow, and yet more preferably whole blood flow, around the modules and through these channels.

In the preferred embodiment, the collagen modules may be first assembled within the enclosure and subsequently covered on their outside by HUVEC by seeding the enclosure with HUVEC. Alternatively, the collagen modules may be first coated with HUVEC and then packed into the enclosure. The interstitial gaps among the modules form interconnected channels that become lined by the endothelial cells, regardless of the method of seeding.

The present invention also provides a method for connecting the tissue construct to a vascular system. Preferably, construction of the tissue construct can be made by using a vascular graft as the enclosure or a combination of inlet and outlet grafts and a separate enclosure to hold the modules. More preferably, conventional anastomotic procedures and/or adopting techniques used in assembling hollow fibre systems into larger units are considered in the invention. Alternatively, adding endothelial cell covered modules to fill a tissue space can lead to perfusion of the resulting construct as the vasculature surrounding the tissue space connects with the endothelial cell lined channels of the construct.

Further aspects of the invention will become apparent upon reading the following detailed description and drawings that illustrate the invention and preferred embodiments of the invention.

To this end, in one of its aspects, the invention provides a new tissue construct having a uniform cell distribution and which is scaleable and can accommodate multiple cell types and in which porosity is created after cell incorporation or embedding.

In another of its aspects, the invention provides a tissue construct wherein said material is non-thrombogenic.

In another of its aspects, the invention provides a method of connecting a tissue construct to a vascular system which consists of constructing a tissue construct using a vascular graft as the enclosure and using a separate enclosure to hold the modules.

Other objects of the present invention will become obvious from the following description taken together with the following drawings.

### BRIEF DESCRIPTION OF THE DRAWINGS

In the drawings, which illustrate embodiments of the invention:

FIG. 1(a) is a schematic drawing of a modular construct design and fabrication.

FIG. 1(b) is a light micrograph of a collagen-HepG2 module before HUVEC seeding.

FIG. 1(c) is a confocal microscopy image of VE-cadherin stained module.

FIG. 1(d) is a modular construct in the flow circuit being perfused with phosphate buffered saline.

FIG. 1(e) is a confocal microscope image of a collagen-HepG2-HUVEC module.

FIG. 2(a) is a chart showing the flow and shear profiles through two collagen modular constructs.

FIG. 2(b) is a MicroCT image of cast of a poloxamine modular construct without HUVEC.

FIG. 3(a) illustrates clot formation times using whole blood studies.

FIG. 3(b) is a chart comparing percentage of initial platelet count over time for fresh whole blood perfused through a HUVEC-covered modular construct.

FIG. 4 is a schematic of modular fabrication with a sieve.

FIG. 5 is a scanning electron micrograph of EC seeded modules one week after seeding.

FIG. 6 shows the structure of poloxamine-methacrylate and methylated poloxamine-methacrylate.

FIG. 7 shows live calcein AM stained HUVEC seeded on methylated poloxamine-collagen modules and film one day after seeding.

FIG. 8 shows Masson trichrome staining of collagen modules.

FIG. 9 shows UEA-1 staining of HUVEC lined channels.

FIG. 10(a) shows a VE-cadherin stained rat aortic EC on collagen modules.

FIG. 10(b) shows CFSE staining of rat microvacular EC at day 11.

FIG. 11 shows bioluminescent images of luciferase transfected CHO cells embedded in HUVEC covered modules in an omental pouch in nude rats at day 7.

### DETAILED DESCRIPTION OF THE PREFERRED EMBODIMENTS

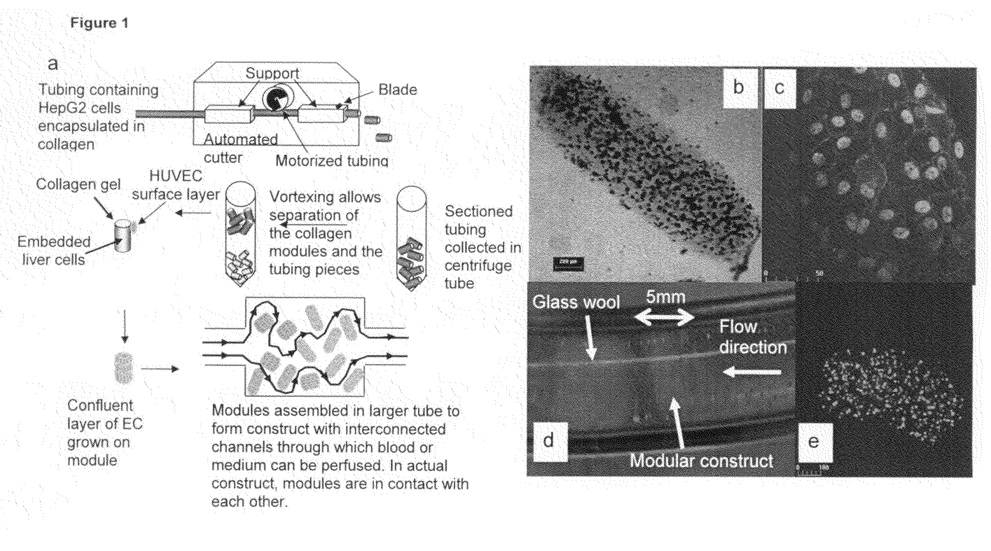

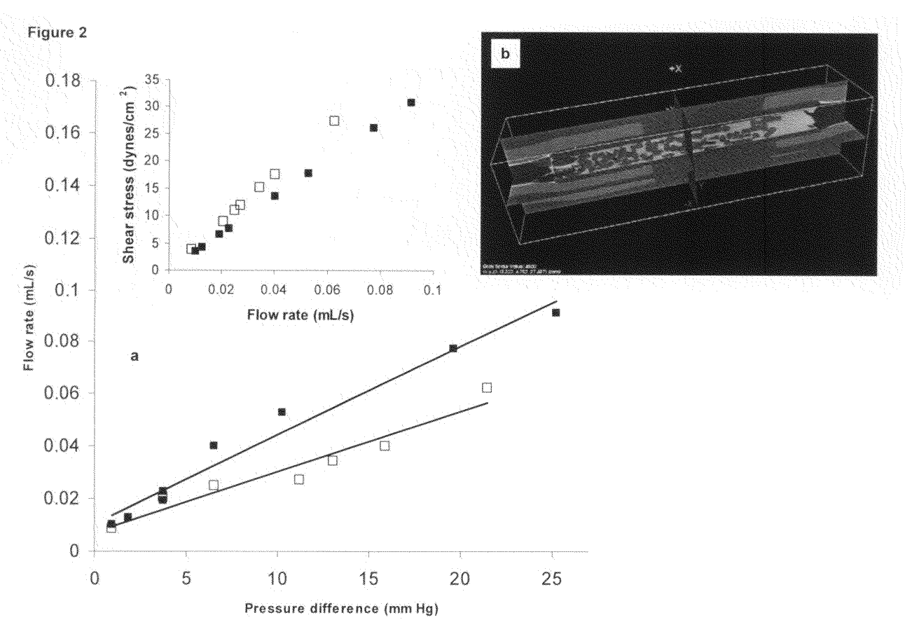

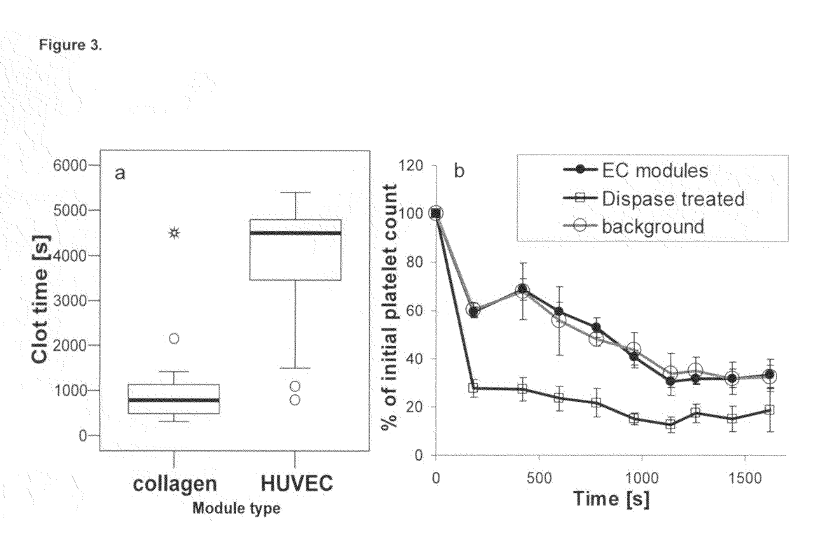

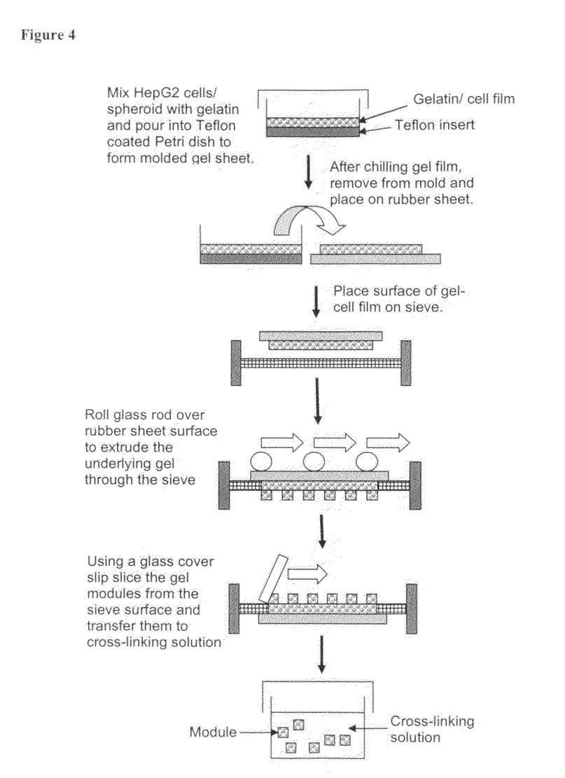

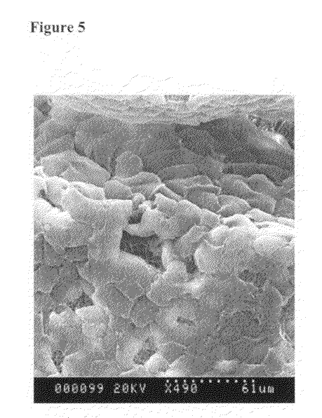

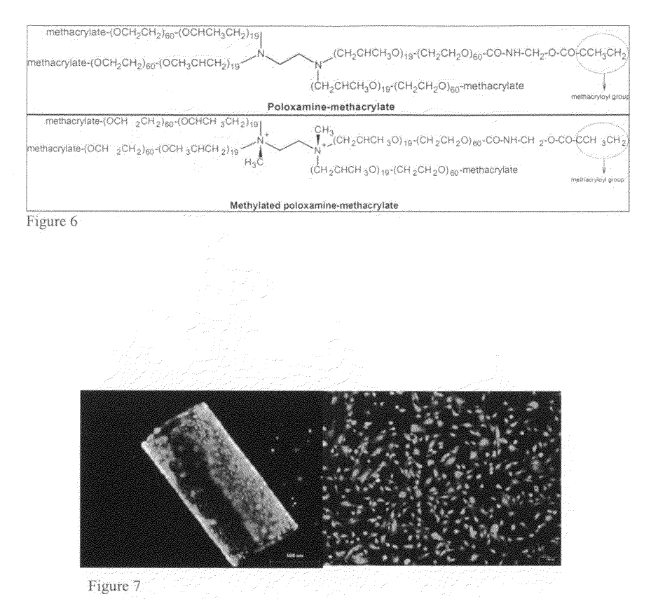

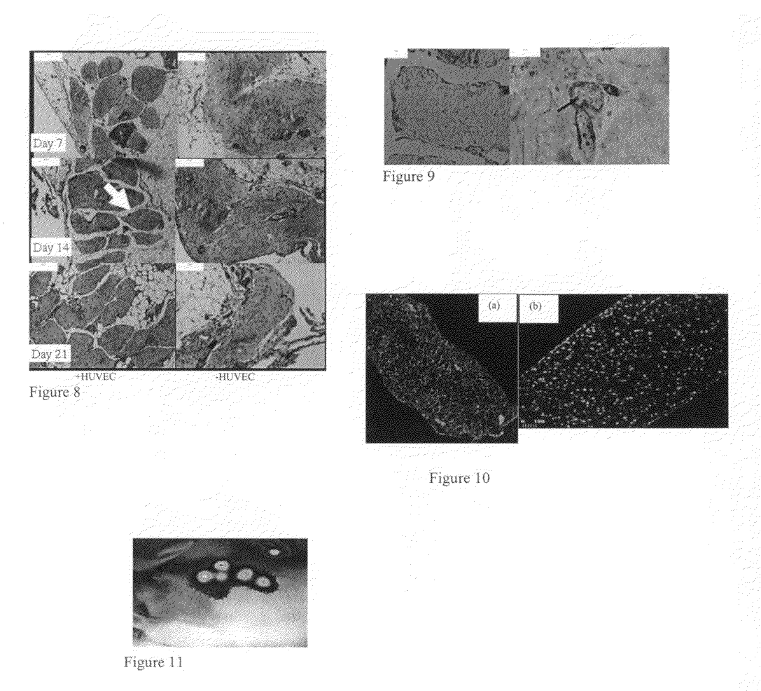

Reference is first made to the figures, a detailed description of which is as follows:
FIG. 1 illustrates modular construct design and fabrication. (a) Collagen with or without HepG2 cells is drawn into the lumen of a sterilized polyethylene (PE) tube and incubated at 37° C. for 30 minutes to allow gelation. The PE tubing containing the gel is fed through an automated tubing cutter and sectioned into 2 mm lengths which are collected in a sterile centrifuge tube. Cell culture medium is added and the tube is vortexed to release the collagen-cell modules from the lumen of the sectioned PE pieces. PE sections float, while collagen modules sink. The collagen cylinders with embedded HepG2 cells are subsequently seeded with HUVEC. Once complete coverage of the collagen surface with HUVEC has been achieved (typically 2-3 days), the cell-seeded cylinders are assembled into a larger structure (here a tube) to form the construct. Assembly of the modules creates a network of interconnected channels that permeate the construct. Medium or blood is perfused through this network to supply nutrients to the cells within the construct. (b) Light micrograph of a collagen-HepG2 module before HUVEC seeding. (c) Confocal microscopy image of VE-cadherin stained module indicating a confluent layer of HUVEC over the module surface at 7 days after seeding. (d) Modular construct in the flow circuit being perfused with phosphate buffered saline. (e) Confocal microscope image of a collagen-HepG2-HUVEC module retrieved from a construct after 7 days of medium perfusion with HepG2 cells labeled with Vybrant CFDA SE.
FIG. 2: (a) Flow and shear profiles through two collagen modular constructs. Flow rate of (PBS) through two separate constructs (construct length, 0.5 cm; construct diameter, 0.3 cm) as a function of applied pressure difference (hydrostatic head); open and filled points represent different constructs. Each point is the mean of two flow rate measurements made at each pressure difference. The slope of the fitted line was used to calculate bed porosity using the Ergun equation from which the shear stress on the surface of the modules was calculated (Insert) for each construct. (b) MicroCT image of microfil cast of a poloxamine modular construct (without HUVEC). Light coloured regions correspond to the microfil (ie the channels) and dark regions correspond to modules, illustrating the interconnectedness of the flow channels that are normally lined with endothelial cells. Porosity based on the number of light pixels was 22.6%.
FIG. 3: Characterization of module thrombogenicity using whole blood studies. (a) Clot formation times. The presence of HUVEC on the modules significantly increased the time to clot formation (p=1.4×10⁻⁵) of slightly heparinized whole blood (0.75 U/mL) in a clotting test. In some cases clot formation never actually occurred and the test was terminated between 4500 and 5400 seconds; in these instances the recorded time was the test termination time. Mean clot time is represented by the thick central line within the box. Open circles and stars represent outliers and extreme outliers respectively. (b) Fresh whole blood (0.75 U/mL heparin) perfused through an HUVEC-covered modular construct (solid circles) maintains platelet levels no different to those measured in the absence of modules (open circles, flow circuit blank; includes polypropylene mesh required to keep modules in place). Blood perfusion through a control modular construct in which HUVEC have been removed by dispase-collagenase action (open squares), however, results in significant reductions in platelet number indicating platelet activation and the thrombogenic response that occurs in the absence of HUVEC. Error bars indicate the standard error of the mean (n=3, 4 and 7 for background, dispase treated modular constructs and HUVEC covered modular constructs respectively).
FIG. 4: Schematic of module fabrication using a sieve.
FIG. 5: Scanning Electron micrographs of BAEC seeded modules 1 week after seeding. Scanning Electron micrographs of BAEC seeded modules one week after seeding showing the classic cobblestone morphology. Good coverage of the modules with EC is achieved after 7 days in culture.
FIG. 6. Methacryloyl groups were added to the ends of poloxamine (poloxamine methacrylate) and then a solution of the poloxamine and collagen was photo-crosslinked to create an interpenetrating network. Greater attachment of EC was obtained with a quaternized (methylated) poloxamine, to which methacrylolyl groups were then added.
FIG. 7. Live (calcein AM) HUVEC seeded on methylated poloxamine-collagen modules (left) and film (right) 1 day after seeding. Stiffness enables shape retention. The methylated poloxamine was combined with collagen (as a semi-interpenetrating network) and this resulted in very good EC attachment to modules.
FIG. 8. Masson trichrome staining of collagen modules in omental pouch in nude rats, showing channels in presence of HUVEC (left) but not in absence of HUVEC (right)
FIG. 9. UEA-1 staining of HUVEC lined channels, day 7, nude rat. Left (7 wk rat); right (5 wk rat; high mag). Arrow shows vessel in cross-section

FIG. 10: (a) VE-cadherin stained rat aortic EC on collagen modules, showing good coverage at day 7 after seeding (b) CFSE staining of rat microvascular EC at day 11. Fibronectin was added to collagen gel to enable EC proliferation
FIG. 11. Bioluminescent images of luciferase transfected CHO cells embedded in HUVEC covered modules in an omental pouch in nude rats at day 7.

### Embodiment 1—Collagen Modules, Automated Cutter

Collagen-HepG2 modules were fabricated (FIG. 1a) by gelling a solution of endotoxin-free collagen, containing suspended HepG2 cells, within the lumen of a small bore polyethylene tube. The tubing was then cut into 2 mm lengths using an automated cutter and gently vortexed to remove the cell-containing collagen modules from the tubing lumen. Modules with different dimensions can be produced by using different tubing diameters and sectioning lengths. HepG2 cell viability within individual modules (FIG. 1b) was greater than 90% and even with perfusion for 7 days (FIG. 1e) viable cell numbers were similar, if not greater than those of modules cultured under static conditions. After 7 days of culture, cell densities reached high values (0.3-1×10⁸ cells/cm³), depending both on cell growth and on module contraction, within an order of magnitude of cell densities within tissues (10⁸-10⁹ cells/cm³).

One day after fabrication, modules were seeded and incubated with HUVEC under static conditions. Full surface coverage and shrinkage was achieved within 3 days (FIG. 1c). In some cases, HUVEC bridging of modules in close proximity was observed. HUVEC densities reached 4.5±1.5×10⁵ cells/cm² (>90% viable) within 7 days, consistent with confluent HUVEC densities observed on tissue culture polystyrene. The quiescent, non-thrombogenic EC lining within the channels of the tissue construct is critical to enable whole blood to percolate around the modules with a significantly lower level of thrombosis than that associated with biomaterial surfaces. HUVEC are useful in this context since they express low basal levels of tissue factor, a potent coagulation initiator. Collagen was selected for the module base material in the prototype as HUVEC naturally reside on a type N collagen membrane, albeit not the same type as that (type 1) which is readily available. It is possible to incorporate other extracellular matrix components, such as type N collagen, elastin peptides and glycosaminoglycans, during module fabrication to further enhance the function of the modules.

Modules were randomly assembled into a tissue construct by pipetting a suspension of modules into a larger tube, acting as the enclosure (FIG. 1d). The modules produced from four meters of collagen filled PE tubing were sufficient to assemble a 0.5-1.0 cm long×0.3 cm diameter construct (construct volume of 0.038-0.075 cm³). The interstitial spaces, formed between the assembled modules, constituted HUVEC-lined interconnected channels, on the order of a few hundred microns in size, that permeated the modular construct enabling fluid and particularly blood perfusion. The seeded endothelial cells were expected to control the dynamic balance of pro- and anti-thrombogenic factors to maintain continuous blood flow without thrombosis. We envision the modular construct, in an appropriate organ-like shape, will be connected to the vascular supply of the host using appropriate host vessels or artificial vascular grafts.

Tissue constructs were perfused at physiological pressure differences (i.e <100 mm Hg) with cell culture medium to simulate the flow of blood through a fully functional construct. Pressure difference versus flow rate flow profiles, obtained for two separate modular constructs (FIG. 2a), were used to estimate construct porosity and shear levels within the channels of the constructs. Analysis of these profiles using the Ergun equation indicated construct porosity was 22% and 24% (lower than expected, see below) in the two constructs shown in FIG. 2a. Using these porosity values, the average shear stress on the HUVEC (FIG. 2 insert) was calculated to be in the range of 3 to 30 dyne/cm² depending on the flow rate. The channels within a similar modular construct (prepared with a stiffer material) are shown in a microCT image in FIG. 2b. This illustrates the interconnectedness of the channels and the laminar, well-defined percolating flow profile in a modular construct. Exposure to flow (24 hours at a flow rate 0.08-0.11 mL/sec/cm²; shear approximately 2-3 dynes/cm²) in the construct increased F-actin levels, and elongated and flattened the HUVEC on the module surface.

The seeded endothelial cells maintained their non-thrombogenic phenotype as demonstrated by various assays, including ones involving whole blood perfusion. The tissue factor activity (factor Xa generation chromogenic assay) of HUVEC seeded modules cultured under static conditions was low. HUVEC covered modules produced significantly longer times to clotting (0.75 U/mL heparin; rocking platform arrangement) than collagen only modules (FIG. 3a, p=1.4×10⁻⁵). In 9 out of 14 trials using HUVEC modules clotting had not occurred at test termination compared to 1 out of 15 trials for collagen only modules. The presence of the HUVEC significantly reduced the thrombogenicity of the module surface.

Lastly and most significantly, slightly heparinized (0.75 U/mL) whole blood was perfused through the constructs at a rate of 0.334 ml/min (equivalent to ˜7 dynes/cm²) and the effluent analyzed for platelet concentration (FIG. 3b). When constructs assembled from HUVEC covered modules were perfused, there was no reduction in platelet concentration relative to the background changes associated with the flow circuit itself (i.e. measured in the absence of modules). Blood perfusion through collagenase-dispase treated HUVEC modules (to remove the HUVEC layer after module shrinkage) significantly reduced platelet concentration in the collected perfusate. The reduction in effluent platelet concentration is an indicator of thrombogenicity in the absence of HUVEC; the absence of this reduction (relative to the background) is an indicator of the functional efficacy of the HUVEC seeded modules in inhibiting platelet activation. Obvious thrombus formation (at 30 minutes) was seen in the majority of flow circuits without endothelial cells, but not when endothelial cells were present. The presence of the HUVEC significantly reduced the thrombogenicity of the construct.

The potential for scaleability arises because, unique to the modular approach, the underlying design principles can be delineated. The three main constraints that influence the design of the modular construct are: nutrient supply; incorporating clinically significant numbers of cells within a construct of implantable volume; and the shear force on the HUVEC layer. Nutrient supply, determined by mass transfer within the construct, was estimated not to be a significant design constraint. Channel dimensions are expected to be of the same size as the modules (i.e., on the order of a few hundred microns) allowing good oxygen mass transfer, the likely limiting nutrient, within the construct channels. Moreover, HepG2 cells remained viable within an assembled construct over 7 days, suggesting mass transfer to the encapsulated cells was sufficient, at least for the cell seeding density and module size used. Since, it has been predicted that a patient could survive on 10% of normal liver function, an engineered liver with the cell densities achieved in our construct (3-10% of tissue densities), could conceivably have sufficient cell mass to support patient survival.

We have demonstrated the use of microscale modular components in a biomimetic fashion to assemble uniform, potentially scaleable (micro)vascularized tissue-engineered constructs containing multiple cell types which were perfused with whole blood. The current prototype enabled maintenance of cell viability, at high cell densities and whole blood perfusion with minimal blood activation. Modular tissue assembly is a biomimetic alternative to traditional scaffold based strategies, which offers many advantages for engineering whole organ and large tissue grafts and potentially transforms the conventional cell seeding/porous scaffold paradigm of tissue engineering.

### Embodiment 2—Gelatin Modules, Hand Cutting

In an alternative embodiment, gelatin modules (˜120 μm diameter×1 mm long) containing HepG2 spheroids were prepared inside a glass micropipette (0.282 mm ID, Drummond microcap) prewashed with Pluronic L101. HepG2 spheroids were prepared by culture in αMEM with serum on bacteriological polystyrene culture dishes for 4 days; at this time spheroids were approximately 100 μm in diameter and contained roughly 300 cells each. Spheroids were suspended in 55 μl of 300 bloom, type A gelatin (25 wt %) liquid (˜40° C.) and a droplet of the gel-spheroid suspension was placed onto a sterilised glass slide, from which it was drawn into the glass micropipette. After 20-30 minutes refrigeration, (enough time to ensure gelation) the gel-spheroid modules were expelled from the glass capillary into a sterile solution of very dilute glutaraldehyde (0.05%) in PBS. After 20 minutes the modules were washed twice in PBS followed by a 1-2 hour wash in cell culture medium. A 20 minute exposure time was sufficient to cross-link the gelatin so that the rods did not fall apart when incubated at 37° C., yet avoided prolonged exposure of the cells to glutaraldehyde. Modules were cut by hand under the microscope into 1 mm lengths, although the automated cutting device used for collagen gel (above) could be used here as well.

Despite the various manipulations and especially the brief exposure to very dilute glutaraldehyde, the cells appeared to remain largely viable based on MTT conversion (the spheroids became purple) and confocal microscopy with the live/dead cell assay, at least for 9 days after fabrication. Not surprisingly the central core of the spheroids remained viable while the outer rim had dead cells, presumably reflecting the effect of the glutaraldehyde.

Modules were randomly packed without difficulty in 2 mm ID PE tubes, capped with a mesh. Packing gelatin rods into the PE tubing (the enclosure) was done by pouring a slurry of the gel rods suspended in PBS (phosphate buffered saline) into the larger diameter tube. A nylon mesh filter (Millipore, pore size 100 μm) was used at the bottom of the tubing to retain the rods; a similar one was mounted on the top to create the tubing construct. Endothelial cells were seeded onto the gelatin rods after loading the rods into the polyethylene tube much as vascular grafts are seeded. The assembled construct was filled with an EC suspension (2-4×10⁵ cells/cm² of gelatin or 1.2-2.5×10⁷ cells/cm³ for a 2 mm diameter×5 cm tube with 70% porosity) and the construct “soaked” in cell suspension for 2 hours to enable the EC to settle and adhere to the gelatin. After gentle rinsing, subsequent static culture for 1 to 4 days was sufficient to reach confluence. The soaking conditions (time, EC concentration) is optimised based on the number of cells retained by the gelatin rods and the uniformity of coverage.

Alternatively, EC were seeded on the gelatin rods prior to assembly into the PE tube (the enclosure) under static conditions in 24 well non-tissue culture plates (to minimise adhesion to the plate itself). The cells are allowed to adhere to the gelatin (1-4 hour incubation) and then cultured to reach confluence over 4 days. Some agitation of the rods within the 24 well plates is needed to obtain reasonably uniform coverage. The cells readily adhered as expected. The EC covered modules were then loaded into the PE tube as above; some loss of EC may occur during this loading step, necessitating a brief incubation (<1 day) to restore a monolayer.

### Embodiment 3—Preparing Modules Through a Mesh

Another means of preparing modules was to push a film of gel containing embedded cells or spheroids, through a 250 μm sieve (FIG. 4). Gelatin modules containing cells or spheroids were produced in this way. Cross-linking (25 minutes, 0.025% GTA) resulted in approximately 200×200 μm×500 gm long rectangular modules and was sufficient to maintain module integrity upon incubation at 37° C. and prevent agglomeration within the culture dish. Gelatin (300 bloom, type A, Sigma-Aldrich Canada, Oakville ON) was cast from a 20 wt % solution at 40° C. onto a teflon disk inserted in a 30 mm petri dish. HepG2 cells or spheroids were added to the gelatin prior to casting (1.4-1.8×10⁷ cells/mL). The cast gel was chilled for 5 minutes at 4° C. in a refrigerator and then removed from the petri dish mold and placed on a rubber sheet (FIG. 4). The cast gel and rubber sheet were then inverted over a 60 mesh (250 μm gap size) stainless steel sieve (WS Tyler, St. Catherines ON) and a glass rod was rolled over the top of the rubber sheet to push the gelatin through the mesh. A glass cover slip was used to collect the extruded gel modules from the lower side of the mesh and transfer them into a 0.025% glutaraldehyde (GTA, Sigma-Aldrich Canada, Oakville ON) aqueous solution. Modules were cross-linked for 25 minutes and then washed 3 times in phosphate buffered saline (PBS, University of Toronto Tissue Culture Media Preparations), incubated in medium for 1 hour and then transferred to fresh medium after which module cultures were fed every two days.

Embedded cells or spheroids remained in the gelatin and no obvious migration out of the modules was observed. In some cases, modules did not separate completely from adjacent ones during the sieve fabrication step. The resulting large agglomerates did not cross-link fully during the GTA treatment and dissolved on incubation at 37° C. Pipetting the modules several times through a disposable 1 mL pipette tip before cross-linking improved module separation and reduced the number of agglomerates. The Hoechst DNA assay indicated that 1 mL of modules contained ˜8.9±0.4×10⁵ cells when a cell suspension was embedded or 5±0.4×10⁴ cells when spheroids were used. Manual counting lead to higher numbers, (˜14.0±1.4×10⁵ cells or 9.1±1.8×10⁵ cells respectively), although of the same order of magnitude.

Glutaraldehyde cross-linking was necessary to generate dimensionally stable modules but it was necessary to minimize GTA exposure in order to minimize the loss of cell viability. We tested the viability of cells encapsulated within gelatin during cross-linking (direct exposure to GTA) and the viability of cells cultured on the surface of gelatin films that had previously been cross-linked with GTA and subsequently washed to remove any GTA solution (Indirect exposure due to residual leakage). For cells embedded in cross-linked gelatin films viability was 11±8%, using CCK-8, after cross-linking with 0.025% GTA for 10-20 minutes. In modules (0.025% GTA, 25 min cross-link) viability was 40±5% relative to non cross-linked modules, measured using the Alamar Blue assay. A more significant loss of viability was seen for spheroids embedded within the gelatin during cross-linking. Using CCK-8, viability was 5-10% for spheroids after cross-linking (0.025% GTA, 10-20 minutes) and was even lower at higher GTA concentrations. Using the Alamar Blue assay, spheroids in modules (0.025% GTA, 25 min cross-linking) had a viability of 30%±1% relative to non-encapsulated spheroids.

Endothelial cells cultured on top of 0.025% GTA cross-linked gelatin films had greater than 80% viability (CCK-8 or Alamar blue) for films cross-linked for 30 min, independent of passage number (P5-P9). Thus leaching of residual GTA did not appear to be a significant problem under the cross-linking conditions (0.025% GTA, 30 min) necessary to stabilize module integrity. The characteristic BAEC cobblestone morphology was observed using SEM (FIG. 5). BAEC adhered well to gelatin films. Centrifugation resulted in detachment of <30% at 400 or 1000 rpm for one clone. Results were slightly different for passage number and were slightly greater at higher cell densities (50,000 cells per well in a 96 well plate). There was no significant difference relative to cells cultured on TCPS. Furthermore the action of pipetting the modules through a pipette tip, which was expected to generate significant shear on the module surface, did not detach the EC or beak up agglomerates of modules bridged by EC, indicating significant adhesion to the gelatin substrate.

### Embodiment 4—Poloxamine Based Materials

In another embodiment modules were prepared using a synthetic collagen-mimetic material that was stiffer than collagen (and therefore resistant to compaction) but that like collagen allows both cell encapsulation and cell growth on the surface. This collagen-mimetic material was a poloxamine-collagen semi-interpenetrating network¹³; poloxamine is a four-arm PEO-PPO block copolymer derivative, Tetronic™ 1107. Methacryloyl groups were added to the ends of the poloxamine (FIG. 6) and a solution of the poloxamine with collagen also in the same solution was photo-crosslinked. Cells (HepG2) were embedded easily and at high viability¹⁵. The poloxamine-collagen material was much stiffer (2,000 to 7,000 Pa for polymer concentrations between 6 to 8%) than collagen alone (˜50 Pa) as was evident also in the cylindrical shape of these modules which was preserved through many weeks of culture. ¹⁵Sosnik A., Leung B., McGuigan A. P. and Sefton M. V., Collagen/poloxamine hydrogels: Cytocompatibility of embedded HepG2 cells and surface attached endothelial cells, Tissue Eng. 11, 1807-1816 (2005)

A positively charged poloxamine hydrogel was also prepared by grafting quaternary ammonium groups in the poloxamine network through a photo-initiated free radical copolymerization of mixtures of poloxamine-methacrylate and ([2-(methacryloyloxy)ethyl]-trimethylammonium chloride (MAETAC)¹⁶. The modification resulted in good HUVEC attachment and confluent monolayers were achieved on films and modules. This material was not suitable for cell encapsulation due to acute cell death associated with exposure to MAETAC during embedding. Following the same quaternization strategy, but with a focus on reducing the cytotoxicity for cell encapsulation by reducing the concentration of reactive methacryloyl derivatives, the tertiary amine groups of poloxamine were methylated with iodomethane—eliminating the need for MAETAC. This derivative was subsequently reacted with methacryloyl isocyanate, producing positively-charged materials (FIG. 6) that were further crosslinkable by a photointiated free radical polymerization¹⁴. A gradual increase of both the storage modulus (G′) and the loss modulus (G″) resulted from increasing the polymer concentration: for example, G′ values were as high as 23,000 Pa for 18% methylated poloxamine-methacrylate hydrogels (at 1 Hz, 100 Pa of oscillatory stress), compared again to ˜50 Pa for collagen gels. HepG2 cells embedded in different compositions and exposed to U.V. light displayed good viability levels after the crosslinking, unlike the MAETAC approach. A well-spread endothelial cell morphology was apparent on methylated poloxamine films after pre-incubation in serum containing medium. The methylated poloxamine was also combined with collagen (as a semi-interpenetrating network) and this resulted in very good attachment to modules (FIG. 7). The methylated poloxamine displays the attributes that make it a useful material for modular tissue engineering. Degradable versions of these modified poloxamines can be prepared by introducing lactic acid groups into the poloxamine prior to the addition of methacroyl groups. This results in stiff poloxamine based modules, that shrink and change in shape (over a few days) as the poloxamine derivative degrades. ¹⁶Sosnik A. and Sefton M. V., Poloxamine hydrogels with a quaternary ammonium modification to improve cell attachment, J. Biomed. Mater. Res. Part A, 75, 295-307 (2005)

### Embodiment 5—Other Embedded Cells

Human umbilical vein smooth muscle cells (UVSMC, a cell-line, ATCC, Manassas, Va.) were embedded in collagen gel modules as described in embodiment 1. The cells were cultured in 10% fetal bovine serum (FBS, Sigma, St. Louis, Mo.) supplemented medium consisting of F-12K Kaighn's modified medium (Gibco, Burlington, ON) further supplemented with 0.1 mg/mL heparin, 0.03 mg/mL endothelial cell growth supplement (ECGS, BD Bioscience, Franklin Lakes, N.J.), 1% penicillin and streptomycin solution (Gibco). To induce UVSMC quiescence, the medium for a confluent UVSMC layer was replaced with quiescence medium (QM), identical to that used above for UVSMC but without serum. Modules containing both UVSMC and HUVEC were cultured in the EGM-2 medium supplemented with the bullet kit and 0.03 mg/mL endothelial cell growth supplement.

Embedded smooth muscle cells (SMC) showed normal morphology (F-actin staining) and protein expression (calponin, SM myosin heavy chain by Western blot). SMC contractile state was sensitive to serum withdrawal¹⁷: embedded SMC phenotype (as determined by presence or absence of serum) affected HUVEC junction morphology (VE-cadherin expression) consistent with the predictions from other SMC-HUVEC co-culture systems¹⁸. SMC phenotype also appeared to affect subsequent HUVEC proliferation rate as assessed by BrdU uptake assay. HepG2 cells were useful as model cells in early studies and the SMC-HUVEC system enabled further exploration of the modules as a co-culture system. ¹⁷B. Leung, MASc thesis, IBBME, University of Toronto (2005).¹⁸A. Armulik, A. Abramsson, and C. Betsholtz, Endothelial/pericyte interactions. Circ. Res. 97, 512-523 (2005)

### Embodiment 6—In Vivo Enclosure

HUVEC covered modules were implanted into an omental pouch, an enclosure to be filled with modules, in nude rats. The omental pouch is prepared by folding the omentum up towards the stomach and suturing (7‘o’ silk sutures) along the left and right edges of the omentum and along the top of the pouch but leaving an opening for the placement of the modules. Modules, suspended in PBS are placed into the omental pouch using a sterile 1000 μL micropipette tip, while preaggregated modules (e.g., prepared by incubation at high density in a small well) are placed into the pouch with tweezers. The opening is sutured closed to completely enclose the modules. FIG. 8 shows that collagen gel modules (in green) coated with HUVEC have channels (see arrow, order of 100 μm in “width”) that persist up to 21 days after implantation in the omental pouch. Without HUVEC the collagen modules remodel and do not appear to form channels. Some of these channels (FIG. 9 right; UEA-1 lectin staining, Ulex Europaeus Agglutinin I, Vector Laboratories) appear to have erythrocytes within the lumen.

To avoid the apparent immune response to xenogeneic EC microvascular rat EC are seeded onto collagen gel modules. A simple modification (inclusion of fibronectin into the collagen gel) has resulted in good rat EC attachment to and junction formation on collagen modules (FIG. 10), although these must be incubated for 11 days instead of 3-7 before confluent modules are obtained.

In order to track the viability of the cells without sacrificing the animal we prepared (by retrovirus) luciferase stably-transfected CHO cells, embedded them in collagen modules, seeded them with HUVEC and implanted them in an omental pouch in the nude rat. Injecting luciferin ip, the Xenogen cooled CCD camera was used to detect, through the skin, the weak emitted light (FIG. 11).

Modules can fill a liver enclosure through intraportal infusion similar to clinical islet transplantation methods¹⁹. In a rat, a midline incision exposes the peritoneal cavity and the underlying portal vein. Modules are loaded into a catheter attached to a 1 mL syringe and injected into the vein via a 25-gauge needle. Manual compression is used to minimize bleeding at injection site. ¹⁹Bottino R, Fernandez L. A, Ricordi C, Lehmann R, Tsan M-F, Oliver R, Inverardi L, Transplantation of allogeneic islets of Langerhans in the rat liver, Diabetes 47, 316-322, 1998

It will be understood that, although various features of the invention have been described with respect to one or another of the embodiments of the invention, the various features and embodiments of the invention may be combined or used in conjunction with other features and embodiments of the invention as described and illustrated herein.

Although this disclosure has described and illustrated certain preferred embodiments of the invention, it is to be understood that the invention is not restricted to these particular embodiments. Rather, the invention includes all embodiments that are functional, electrical or mechanical equivalents of the specific embodiments and features that have been described and illustrated herein.

### Methods for Collagen Gel Prototype [Embodiment 1]

### Cell Culture

The human hepatoma cell line, HepG2 (American Type Culture Collection, Rockville, Md.) was cultured in 25 cm² tissue culture flasks in RPMI 1640 culture medium with L-Glutamine (Invitrogen Canada, Burlington, ON) supplemented with 15% bovine calf serum (Hyclone, Logan, Utah) and 2% penicillin and streptomycin (Invitrogen Canada, Burlington, ON) at 37° C. in a 5% CO₂/95% air humidified atmosphere. Human umbilical vein endothelial cells (HUVEC, Cambrex Bio Science Walkersville, Inc), were cultured in 75 cm² tissue culture flasks in EGM-2 medium suggested by the suppliers supplemented with EGM-2 bullet kit (Cambrex Bio Science, Walkersville, Inc) at 37° C. in a 5% CO₂/95% air humidified atmosphere. In modules where both cell types were present, both cell types were cultured in HUVEC culture medium.

### Module Fabrication

Vitrogen collagen solution (Type I, bovine dermal, 3.1 mg collagen per mL; Cohesion technologies, Palo Alto, Calif.) was mixed with 10× minimum essential medium (Invitrogen Canada, Burlington, ON, 125 μL 10× medium per mL collagen) and neutralised using 0.8 M NaHCO₃ (Sigma-Aldrich Canada, Oakville, ON). Pelleted HepG2 cells were mixed with the neutralised collagen (2×10⁶ cells/mL) and the solution drawn into the lumen of an ethylene oxide gas sterilized polyethylene tube (0.76 mm ID×1.22 mm OD) connected to a syringe at one end. After 30 minutes incubation, to allow collagen gelation, the gel-filled tubing was cut into 2 mm lengths using a custom-built automated cutter (FIG. 1a, FCS Technology, London ON). Sections were vortexed gently in cell culture medium to remove the gel-cell module cores from the tubing lumen. The collagen-cell modules were allowed to settle, separated from the polyethylene tubing and cultured in petri dishes under static conditions. Collagen only modules were fabricated identically (same collagen concentration) without the addition of the HepG2 cell pellet.

### Endothelial Cell Seeding

HUVEC (P1-6, 1.5-2.0×10⁶ cells per mL of settled modules) were added to modules with or without encapsulated HepG2 cells in a 15 mL centrifuge tube and incubated for 60 minutes with gentle shaking every 10 minutes. Modules were then transferred into a non-tissue culture polystyrene petri dish. Medium was replaced every 1-3 days.

### Module Dimensions

After incubation overnight a sample (n=96) of modules containing HepG2 cells was selected and light microscopy images were taken of each module in a 96 well plate (one module/well) using an Olympus microscope. Modules were then seeded with approximately 1.5×10⁶ HUVEC per mL of settled modules, and incubated for 4 days, after which they were re-imaged. Measurements of module diameter and length, before and after endothelial cell seeding, were made using ImagePro software (Media Cybernetics, San Diego Calif.).

### Cell Viability and Enumeration Within Modules

Cell metabolism of encapsulated cells was measured using the Alamar blue (AB) assay at days 1, 3 and 7. Briefly a micropipette was used to add 10 modules (3 replicates), containing HepG2 cells, in a 200 μL volume, into a 24 well plate. 10% AB (BioSource International, Inc. Camarillo, Calif.) was added and the sample incubated for 7 hours. Supernatant samples were transferred into a 96 well plate and read using a Sunrise ELISA plate reader (Tecan, Maennedorf, Switzerland) at 570 nm and 600 nm. Module samples were then digested using collagenase (Sigma-Aldrich Canada, Oakville, ON, final concentration 0.236 mg/mL in culture medium), incubated overnight and stained with trypan blue. The numbers of live and dead cells were counted manually using a hemocytometer.

To assess cell viability, within the assembled construct, modules containing HepG2 cells or collagen only modules seeded with HUVEC were cultured under static conditions for 6 days and then within a flow circuit (see below) for 24 hours. Modules were retrieved from the circuit and tested immediately for viability by digestion and staining with trypan blue as above. The viability of HepG2 cells cultured within an assembled construct for 1 week was assessed using Vybrant CFDA SE prelabelled cells (10 μM, carbofluorescein diacetate succinimidyl ester, Molecular Probes, Burlington, ON). One day after fabrication the HepG2 modules were seeded with HUVEC and then after 2 days incubation, to allow module shrinkage, were assembled into a construct within a flow circuit. After 1 week of medium perfusion modules were retrieved from the flow circuit, fixed in 3.7% paraformaldehyde-PBS (Electron Microscopy Science, Hatfield, Pa.) for 30 minutes, washed in PBS and observed using fluorescence microscopy (Zeiss, Axiovert 135).

### Construct Assembly and Flow Circuit Perfusion

Fifty mL centrifuge tubes with two holes punctured in the cap through which to thread Masterflex L/S-13 and L/S-16 tubing (Labcor, Anjou, QC) and approximately 0.015 g of glass wool (˜0.075 cm³) (to hold the modules in place) were assembled into a continuous loop flow circuit with a number of other connectors and stopcocks (various suppliers). A Masterflex peristaltic pump was used to circulate medium through the flow loop from a 19 mL reservoir. Modules (0.5-1.0 mL) were loaded into the circuit (total circuit volume 20 mL), within a laminar flow hood, using a 10 mL pipette via a luer lock connector. Modules were maintained in the flow circuit at 37° C. in a 5% CO₂/95% air humidified atmosphere for 24 hours or 1 week. Medium was added to the reservoir every 1-2 days.

### Flow Profile Measurements and Porosity Determination

Pressure difference across the construct was recorded using low pressure gauges (H.O. Trerice, Oak Park, Mich.), inserted on either side of the construct. Duplicate measurements of flow rate through the construct were measured for a range of pressure differences, by the timed collection of 0.5 mL medium from the circuit via a T-connector output. The gradient of flow rate versus pressure difference (Darcy's permeability) was calculated and the gradient of similar curves measured in the absence of a construct (i.e., with glass wool only present) was subtracted to isolate the pressure difference contribution from the construct. The Ergun equation was then solved for porosity by iteration using the Solver program in Microsoft Excel. The values used for constants present in this equation were, length of construct 0.5 cm, fluid viscosity 0.01 g/cm³, module diameter 0.0411 cm and shape factor 0.874.

A construct of the same diameter and length was prepared using modules of a stiffer material (20% poloxamine-collagen modules¹³), and so enable perfusion with the viscous microfil solution (“low viscosity”, Flow Tech, Inc. Carver, Mass.; component:diluent ratio of 4:15, 10% curing agent) used for microCT (Mice Imaging Centre (MiCe), Hospital for Sick Children, Toronto). Using Microview software the number of pixels above the threshold corresponding to the microfil was used to calculate the volume fraction of microfil and hence the construct porosity.

### Clotting Time

Fresh whole blood (10 mL) was collected from consenting donors (with ethics approval by the University of Toronto), who had not taken medication within 72 hours of phlebotomy, into a syringe containing heparin (final concentration 0.75 U/mL), after discarding the first mL. A 350 μL sample of slightly heparinized blood was mixed with 200 μL of collagen modules or HUVEC coated modules in a microcentrifuge tube. A 400 μL sample of this was then pipetted into a 25 cm length of polypropylene tubing (1.57 mm ID) connected at either end via Silastic™ tubing (1.57 mm ID) to 200 μL pipette tips connected to a rocking platform²⁰. Rocking was initiated and the time until blood motion ceased or significant clot deposition occurred within the tubing was recorded as the clotting time. ²⁰Gemmell, C. H., Ramirez, S. M., Yeo, E. L., Sefton, M. V. (1995) J. Lab. Clin. Med. 125, 276-287.

### Construct Perfusion

Constructs were assembled from HUVEC covered modules or HUVEC covered modules treated for 15 minutes in 100 mg/mL collagenase dispase solution (Roche, Mississauga, Ontario) to remove all HUVEC from the surface (for control modules), yet retain the size and stiffness of the contracted collagen. The short treatment time ensured module dissolution did not occur and microscope observation confirmed removal of the HUVEC layer. Constructs were assembled within a 0.2 mL length of a 1 mL graduated pipette and held in place at both ends with 1 cm² sections of polypropylene mesh (PPM-3, Biomedical Materials, Slatersville, R.I.). Silastic™ tubing (10 cm, 3.18 mm ID) was used to connect the pipette section to the syringe pump (824E Infusion pump model A-99, Razel Scientific Instruments Inc., Fairfax Vt.). The construct was pre-filled with PBS to prevent air bubble formation.

Blood was collected in a 10 mL syringe from consenting volunteers that had taken no medication into 0.75 units/mL heparin. It was necessary to use a small amount of heparin (0.75 U/mL is much less than the 5 U/mL needed to stop all coagulation) to prevent premature clotting during the blood draw or while the blood was sitting in the syringe pump. The Silastic™ tubing was filled with blood from the syringe before being connected to the PBS pre-filled pipette/construct section. The syringe was then placed on the syringe pump located on a rocking platform (to minimize blood settling) within a 37° C. oven and blood was perfused through the construct at a rate of 0.334 mL/min. At regular intervals during perfusion, 400 μL it samples of the perfusate were collected in 0.6 mL graduated microcentrifuge tubes containing 8 μL of 200 mM EDTA. An initial sample was collected from the syringe before connecting it to the construct. The experiment was terminated when all the blood from the syringe had been used or if circuit blockage occurred. Constructs were removed and dissected for evidence of thrombus formation within the construct or the polypropylene mesh.

### Statistics

The Students t-test was used to determine significant difference when only 2 treatment groups were being compared. Analysis of variance (ANOVA) was used to test for significant differences among multiple test groups. Q-Q plots were used to assess the normality of the data. The Levene's test for homogeneity was used to test for equal variance among samples. When equal variance could be assumed the Tukey HSD post-hoc test was used to identify significant differences among multiple test groups. When equal variance could not be assumed the Games-Howell post-hoc test was used to identify significant differences among multiple test groups. In all tests were two-tailed and a p-value of 0.05 was considered significant.

### Methods for Embodiment 3

### Cell Culture

The human hepatoma cell line, HepG2 (American Type Culture Collection, Rockville, Md.) was cultured in 25 cm² tissue culture flasks using α-MEM culture medium (University of Toronto Tissue Culture Media Preparations) supplemented with 10% fetal bovine serum, 100 U/mL penicillin and 100 ng/mL streptomycin (Invitrogen Canada, Burlington ON), at 37° C. in a 5% CO₂/95% air humidified atmosphere. Spheroid cell aggregates were formed by the addition of 0.5 mL of cell suspension (3×10⁶ cells/mL) to 4.5 mL cell medium in 60 mm non-tissue culture polystyrene dishes, (Fisherbrand). Bovine aortic endothelial cells (BAEC) were harvested using the method of Jaffe et al. (²¹) in the laboratory of Dr. P. Marsden, and cultured on 60 mm tissue culture polystyrene dishes coated with 0.2% gelatin (Sigma-Aldrich Canada, Oakville, ON) using RPMI 1640 culture medium with L-Glutamine (Invitrogen Canada, Burlington ON) supplemented with 15% bovine calf serum (Hyclone, South Logan, Utah) and 10% penicillin and streptomycin at 37° C. in a 5% CO₂/95% air humidified atmosphere. Within a week of cell harvest from the animal, single clones were selected using a cloning tube and then expanded. Cells were fed every two days and sub-cultured once per week with a 5:1 splitting ratio. Two or three clones, each from different aortas, were used for each set of experiments to highlight differences among clones. ²¹C. A. Jaffe, R. L. Nachman, C. G. Becker, C. R. Minick, Culture of human endothelial cells derived from umbilical veins: identification by morphology and immunological criteria, J. Clin. Invest. 52, 2745 (1973).

### Module Characterization

The cell density within the modules was determined by measuring the amount of DNA in proteinase K digested gelatin samples using Hoechst 33258 (Molecular Probes, Eugene, Oreg.) with a Gemini XS fluorescent plate reader. Gelatin modules were frozen in cryovials using liquid nitrogen, freeze dried and digested in proteinase K solution (0.5 mg/mL proteinase K and 0.1 mg/mL SDS in a buffer solution of 50 mM tris-HCL, 0.1 M EDTA, 0.2 M NaCl, pH 7.4) for 15 h at 55° C. with gentle shaking. Samples were aliquoted with Hoechst dye solution (10 mM Tris, 1 mM EDTA.Na₂2H₂O, 0.2M NaCl, 0.1 μg/mL Hoechst 33258 dye) in equal volumes into a black fluorescence plate and fluorescence was read at an excitation wavelength of 360 nm and an emission wavelength of 465 nm. Samples containing known numbers of cells were used for calibration. DNA content was also calibrated with calf thymus DNA (Sigma-Aldrich Canada, Oakville ON). For comparison cell density was also determined manually by digesting gelatin modules with collagenase (1.25 U/mL, Molecular Probes, Eugene, Oreg.) at 37° C. for 6-12 h. The released cells or spheroids were pelleted, washed in PBS, incubated with trypsin and counted using a hemocytometer.

Cell viability was assessed using either the tetrazolium based CCK-8 assay (Dojindo Molecular Technologies, Inc., Gaithersburg, Md.) or Alamar Blue Assay (AB, BioSource International, Inc. Camarillo, Calif.). Briefly, the test solution was added to the cells, (10% test solution for both CCK8 and AB) and incubated for 3 h (CCK-8) or 6.5 h (AB). Aliquots of the medium were transferred into a fresh 96 well plate and solution absorbance read in a Versa max plate reader at 450 nm (CCK-8) or 570 nm and 600 nm (AB).

### Effect of Glutaraldehyde (GTA) on Cell Viability

The viability of cells cultured on cross-linked gel films was assessed to evaluate the indirect effect of any long term GTA leaching from the gels. Gelatin films, (cross-linked with different GTA concentrations and reaction times), were washed 3 times in PBS, incubated for 1 h in medium and then incubated in fresh medium overnight. BAEC or HepG2 cells were seeded on the cross-linked films at densities between 5×10³ and 50×10³ cells per well and incubated overnight. Viability was measured using CCK-8 or Alamar blue and reported relative to tissue culture polystyrene (TCPS).

The direct effect of GTA was assessed by measuring the CCK-8 viability of HepG2 cells (as a suspension or as spheroids) within gelatin films which were cross-linked with different GTA concentrations for different times while the cells were present. The effect of GTA cross-linking on a gelatin module (not a film) was tested using Alamar blue. Viability was calculated relative to monolayers of cells on TCPS or to non cross-linked modules.

Seeding Modules with BAEC

BAEC (1-2×10⁶ cells) were added to 5 mL of settled gelatin modules (no HepG2 cells) suspended in culture medium in a 60 mm non-tissue culture polystyrene petri dish. Cultures were incubated and observed daily using optical microscopy. After 7 days or when a confluent layer of EC had formed, modules were prepared for SEM analysis using a method slightly modified from one described previously by Wissemann et al. (²²). Modules were washed in PBS, fixed in 4% GTA on ice for 1 h and then transferred to a gelatin coated glass cover slip. After a 1 h treatment on ice with 10% GTA, samples were serially dehydrated in ethanol, frozen in liquid nitrogen and freeze dried. After gold coating, samples were examined using a Hitachi S-570 scanning electron microscope at an accelerating voltage of 20 kV. ²²K. W. Wissemann, B. S. Jacobson, Pure gelatin microcarriers: synthesis and use in cell attachment and growth of fibroblasts and endothelial cells, In Vitro Cell. Devlop. Biol. 2, 391 (1985).

### BAEC Adhesion and Growth

BAEC growth on gelatin films was assessed both manually and using the AB assay. BAEC were seeded (10-40×10³ cells per well) on TCPS and gelatin films (cross-linked for 30 min or 120 min with 0.025% GTA) in 96 well plates and incubated either overnight or for 5 days prior to manual counting (after trypsinization) or AB assay.

The adhesion strength of BAEC to gelatin films was compared to that on TCPS using a centrifugation assay. BAEC (P5 to P8) were seeded at 10 to 25×10³ cells/well (96 well plate) and incubated overnight to effect adhesion. Wells were then washed and filled with PBS. Pressure sensitive film was used to seal the PBS within the wells and the plates were inverted. CCK-8 was used to quantify the number of cells per well after centrifugation at 400 or 1000 rpm for 6 min. Adherence was calculated by comparison with a static control.

### Statistics

ANOVA analysis was used to test for significant differences (p<0.05) in experimental parameters for multiple test groups. Post hoc analysis was performed using the Tukey Honest Significant Difference test (α=0.05). The student t-test (p<0.05, 2 tailed) was used to test for the significance between groups when only 2 test groups were being compared.

### Methods for Embodiment 5

### Module Fabrication and Cell Seeding

Modules were fabricated as in embodiment 1. Briefly, acidified bovine type I collagen solution (3 mg/mL, Vitrogen™, CohesionTech, Palo Alto, Calif.) was mixed with 10× MEM medium (Gibco) and neutralized with sodium bicarbonate solution (Sigma), UVSMC were trypsinized and resuspended in the neutralized collagen solution at the desired cell density and the mixture was drawn into gas sterilized polyethylene tubing (Becton Dickson, Intramedic™ brand, PE60, I.D./O.D.=0.76 mm/1.22 mm) and incubated at 37° C. for 1 hour. Upon gelation, the tubing was removed from the incubator and cut into 2 mm sections, using an automated custom built cutter. These sections were collected in a 50 mL polypropylene centrifuge tube containing 30 mL of UVSMC culture medium. The collagen modules were then separated from the tubing by gentle vortexing and were collected at the bottom of the centrifuge tube. Collagen-only modules were fabricated similarly without the addition of cells.

For endothelial cell seeding, 2 million HUVECs were used for every 4 meters of tubing prepared, equivalent to approximately 10 mL of settled modules. Trypsinized HUVECs were mixed in 10 mL of HUVEC medium in a 15 mL centrifuge tube with modules. The centrifuge tubes were left at room temperature on a uniaxial rocker (Bellco Biotechnology, Vineland, N.J., cat #7740-10010) for 30 minutes, after which the modules were transferred into a 100 mm non-tissue culture treated Petri dish and left in the incubator for 24 hours. Modules were transferred to a new non-tissue culture treated Petri dish 24 hours post seeding and cultured with fresh HUVEC medium, with or without ECGS depending on the presence or absence of UVSMC, respectively, for up to 14 days.

### Immunofluorescence Staining and Confocal Imaging

To prepare for immunofluorescence imaging, modules were removed from the culture medium, rinsed with PBS (pH=7.4) and fixed for 30 minutes in 4% paraformaldehyde solution (Sigma), followed by incubation in 0.2% Triton X-100 solution for 4 minutes (to permeabilize the cell membrane) and PBS rinses (3×10 minutes). The samples were incubated with primary antibodies at 1:50 dilution in PBS, in the dark at room temperature for 30 minutes. To assess HUVEC junction morphology, the modules were stained with anti-human VE-cadherin polyclonal rabbit IgG (Sigma). Smooth muscle cell phenotype was characterized by staining with anti-human smooth muscle alpha-actin monoclonal mouse IgG (Sigma). FITC conjugated anti-BrdU monoclonal mouse IgG (Santa Cruz Biotechnology, Santa Cruz, Calif.) was used to assess cell proliferation (see below). After primary antibody incubation, the samples were washed with PBS (3×10 minutes) followed by complementary secondary antibody staining at 1:200 dilution in PBS for 30 minutes. Secondary antibodies with AlexaFluor™ dyes were purchased from Molecular Probes, Burlington, ON (AlexaFluor™ 488 goat anti-rabbit IgG and AlexaFluor™ 568 goat anti-mouse IgG).

Samples were visualized using a BioRad laser scanning confocal microscope (Max-Bell Research Centre, UHN, Toronto, ON, model MRC-1024ES) equipped with a motorized Z-plane stage. Control of the laser scan head and data collection were managed by the software package provided by BioRad (LaserSharp, version 3.2). Typically 20 z-section slices were collected and projected for each composite image. All images were exported as uncompressed .tiff format files for data analysis.

### Cell Viability

To determine the viability of both embedded UVSMC and surface seeded HUVEC in collagen modules, a two color Live/Dead™ Assay was used (Molecular Probes). All procedures were performed following manufacturer recommendations. The staining solution consisted of 4 μM of calcein-AM and 4 μM of EthD-1 in 1× PBS. Modules were incubated (5% CO₂, 95% air, 100% humidity) at 37° C. for 20 minutes in staining solution followed by PBS rinses (3×10 minutes). Stained modules were visualized using confocal microscopy as described above.

Proliferation of embedded and surface seeded cells was determined using 5-bromo-2′-deoxyuridine (BrdU) incorporation assay. Stock solution of BrdU was prepared by dissolving BrdU powder (Molecular Probes) in sterile dimethyl sulfoxide (DMSO, Sigma) at a concentration of 10 mM. Modules were incubated in a 1:1000 dilution of BrdU stock solution in EC medium for 2 hrs (final concentration of BrdU is 10 μM), followed by immunofluorescent analysis as above. Cells were counterstained with propidium iodide at 10 μg/mL in 1× PBS (Sigma), so that proliferating cells were double stained for convenient identification.

1. A new tissue construct having a uniform cell distribution and which is scaleable and can accommodate multiple cell types and in which porosity is created after cell incorporation or embedding.

2. A new tissue construct as claimed in claim 1 which consists of an enclosure randomly filled with discrete and separable components.

3. A new tissue construct as claimed in claim 2 wherein said enclosure is a column, a tube or a tissue space.

4. A new tissue construct as claimed in claim 2 which contains a plurality of channels created from a plurality of interstitial spaces or voids created by said components.

5. A new tissue construct as claimed in claim 4 wherein said interstitial spaces or voids are interconnected forming a plurality of interconnected channels through said construct.

6. A new tissue construct as claimed in claim 5 where said channels are narrow.

7. A new tissue construct as claimed in claim 4 which has a porosity of from 0.3 to 0.99 where the porosity is defined as the ratio of volume of interstitial space to the volume of the construct.

8. A new tissue construct as claimed in claim 7 which ranges from a mm to several cm and has a tissue specific function of said multiple cell types embedded within the components.

9. A new construct as claimed in claim 2 wherein said components contain tissue-specific cells embedded within a material that forms said discrete components.

10. A new construct as claimed in claim 9 wherein said components are cylindrically or spherically shaped.

11. A new construct as claimed in claim 10 wherein said components are less than a mm in critical dimension.

12. A new construct as claimed in claim 10 wherein said components are less than 500 um in critical dimension.

13. A new construct as claimed in claim 10 wherein said components are less than 250 um in the critical dimension.

14. A new tissue construct as claimed in claim 1 which is perfusable.

15. A porous, perfusable tissue construct as claimed in claim 14 consisting of a plurality of modules of cell compatible material which provides adequate dimensional stability to each module within an enclosure and a plurality of interconnected channels.

16. A new tissue construct as claimed in claim 15 wherein said modules are made of an inherently non-thrombogenic material.

17. A tissue construct as claimed in claim 15 wherein said modules consist of a tissue-specific cell or a tissue-specific cell aggregate or tissue fragment embedded within a homogeneous gelatinous material.

18. A tissue construct as claimed in claim 17 wherein said gelatinous material is collagen or gelatin.

19. A tissue construct as claimed in claim 16 wherein said module is formed by suspending cells in a liquid matrix material and subsequently solidified.

20. A tissue construct as claimed in claim 16 wherein said module is preformed as a single porous entity and then filled with a multiplicity of cells.

21. A tissue construct as claimed in claim 16 wherein said module is formed by encapsulating cells in an appropriate material so that the cells are suspended in an aqueous phase in the core of a capsule and the material is used to form a semi-permeable shell.

22. A tissue construct as claimed in claim 16 wherein said modules are cylindrical or spherical shaped.

23. A tissue construct as claimed in claim 16 wherein said modules are hollow cylinders or saddle-shaped.

24. A tissue construct as claimed in claim 15 wherein the modules are covered with endothelial cells and the material of said modules adheres said endothelial cells to its surface.

25. A tissue construct as claimed in claim 15 wherein the modules are covered with endothelial cells which do not completely fill the interstitial spaces between said modules thereby allowing fluid to flow through said channels.

26. A tissue construct as claimed in claim 24 where a pseudo-capillary network is created capable of supporting blood perfusion through said channels.

27. A tissue construct as claimed in claim 15 wherein said modules consist of a cell compatible material that provides dimensional stability to said module and prevents agglomeration of tissue-specific cells into a single cellular mass without interconnected, perfusable channels.

28. A tissue construct as claimed in claim 27 wherein said cell compatible material is selected from the group consisting of agarose, alginate, collage, polyacrylates and stable synthetic biocompatible polymers.

29. A tissue construct as claimed in claim 28 wherein said polymer is selected from the group consisting of collagen-poloxamine, photo-crosslinkable polyethylene glycol based materials and gelatin.

30. A tissue construct as claimed in claim 27 wherein said modules are coated with a second material to enhance the attachment of said endothelial cells.

31. A tissue construct as claimed in claim 30 wherein said second material is selected from the group consisting of collagen, protein, cross-linking agents

32. A tissue construct as claimed in claim 15 where said enclosure is the walls of a tissue cavity.

33. A porous and liquid perfuseable tissue construct as claimed in claim 15 consisting of tissue-specific cells embedded in short collagen gel cylinders or spheres onto which endothelial cells are adhered, and randomly packed into an enclosure and channels formed by interconnected interstitial spaces.

34. A tissue construct as claimed in claim 33 wherein said tissue specific cells are selected from the group consisting of liver cells, islets of Langerhans, cardiac muscle cells and fat cells.

35. A tissue construct as claimed in claim 33 wherein said cylinders have a diameter of from 50 to 500 um and a length of from 250 um to 2 μmm.

36. A tissue construct as claimed in claim 33 wherein said endothelial cells are human umbilical vein endothelial cells.

37. A tissue construct as claimed in claim 33 wherein larger diameter modules are packed proximal and distal to the pseudo-capillary bed inside said enclosure.

38. A tissue construct as claimed in claim 33 wherein said enclosure contains mixtures of modules with cells of different cell types.

39. A tissue construct as claimed in claim 16 wherein said enclosure is a vascular graft or a combination of inlet and outlet grafts and a second enclosure to hold the modules.

40. A tissue construct as claimed in claim 27 wherein said material is non-thrombogenic.

41. A method of connecting a tissue construct to a vascular system which consists of constructing a tissue construct as claimed in claim 16 using a vascular graft as said enclosure, and using a separate enclosure to hold said modules.

42. A method as claimed in claim 42 wherein said modules are covered with endothelial cells.

43. A new, scaleable tissue construct having a uniform cell distribution and which can accommodate multiple cell types consisting of a plurality of discreet and separable modules.

44. A new, scaleable tissue construct as claimed in claim 43 wherein said modules are non-agglomerating cell aggregates.

45. A new, scaleable tissue construct as claimed in claim 44 wherein said aggregates are produced without an imbedding material and in such a way that each aggregate repels the others and prevents their agglomeration in a large, non-profusable construct.
